(19) United States
(12) Patent Application Publication
(10) Pub. No.: US 2023/0330107 A1
(43) Pub. Date: Oct. 19, 2023

(54) METHODS TO PREVENT HIGH-GRADE SEROUS OVARIAN CANCER AND BREAST CANCER BY ADMINISTERING ANTIPROGESTINS

(71) Applicant: The Trustees of Indiana University, Indianapolis, IN (US)

(72) Inventor: Jaeyeon Kim, Indianapolis, IN (US)

(73) Assignee: The Trustees of Indiana University, Indianapol, IN (US)

(21) Appl. No.: 18/042,931

(22) PCT Filed: Aug. 25, 2021
(86) PCT No.: PCT/US21/47499

§ 371 Date: Feb. 24, 2023

Related U.S. Application Data
(60) Provisional application No. 63/072,414, filed on Aug. 31, 2020.

(51) Int. Cl. A61K 31/567; A61K 31/57; A61P 35/00

(52) U.S. Cl. CPC A61K 31/567; A61K 31/57; A61P 35/00; A61K 2039/505

(57) ABSTRACT

Disclosed are methods to prevent an occurrence of a high-grade serous ovarian cancer and/or breast cancer in a subject by administering a therapeutically effective amount of an antiprogestin to the subject.

This Application claims the benefit of U.S. Provisional Application No. 63/072,414, filed on Aug. 31, 2020, which is incorporated herein by reference in its entirety.

### STATEMENT OF GOVERNMENT SUPPORT

This invention was made with government support under CA179137 awarded by National Institutes of Health. The government has certain rights in the invention.

### BACKGROUND

Among the ovarian cancers, high-grade serous ovarian cancer (HGSOC), also known as high-grade serous carcinoma (HGSC), is the most common and deadliest ovarian cancer type, and accounts for over 70% of ovarian cancer cases and deaths. Breast cancer is the most diagnosed cancer among women worldwide. It is the most frequent cancer and is the leading cause of death from cancer in women. Advanced ovarian and breast cancers, which have already metastasized, are rarely curable. Thus, prevention is critical to reducing mortality from these cancers.

Cancer prevention is especially an urgent matter for women carrying a deleterious germ line BRCA1 or 2 mutation. These BRCA1/2-mutation carriers have extraordinarily high lifetime risks of developing predominantly ovarian (10-60%) and breast cancer (60-80%). Although not all of these high-risk women develop these cancers, these BRCA-mutation carriers are advised to undergo prophylactic surgeries at a young age (under 40-45) to reduce cancer risk.

Thus, there is a pressing need to devise an effective, non-surgical prophylactic option for ovarian and breast cancer prevention. The present invention addresses this need.

### SUMMARY

In one aspect, the disclosure relates to methods to prevent an occurrence of a high-grade serous ovarian cancer in a subject by administering a therapeutically effective amount of an antiprogestin to the subject.

In another aspect, the disclosure relates to methods to prevent an occurrence of a breast cancer in a subject by administering a therapeutically effective amount of an antiprogestin to the subject.

Additional embodiments, features, and advantages of the disclosure will be apparent from the following detailed description and through practice of the disclosure.

### BRIEF DESCRIPTION OF THE DRAWINGS

FIGS. 1A-1I show that the PR antagonist mifepristone suppressed HGSC development and extended mouse survival. FIGS. 1A-1B show the tumor phenotype of DKO mice treated with placebo. Overall, placebo-treated DKO mice developed HGSC in the fallopian tube (PT, yellow arrows) along with abundant peritoneal metastases to the omentum (red arrows), mesentery (blue arrows), peritoneum (white arrows), and diaphragm (green arrows). A mouse shown was sacrificed at 7.3 months of age (FIGS. 1A-1B). Scale bar, 0.5 cm. FIGS. 1C-1H show representative tumor phenotype and histology of DKO mice treated with mifepristone (RU486). Typically, mifepristone-treated DKO mice developed little peritoneal metastasis: small tumor nodules in the omentum (a red arrow) from a 19-month-old DKO mouse treated with mifepristone (3 mg for 3 months). Scale bar, 0.5 cm (FIGS. 1C-1D). Histologically, the fallopian tubes contain multiple cysts (FIG. 1E), necrotic and fibrous tissues (FIG. 1F), proliferative stromal cells (FIG. 1F), and a small segment of carcinoma cells (FIG. 1E, cells inside a dotted circle), along with a small group of HGSC cells in the omentum (FIG. 1G). Low-grade endometrioid-type carcinoma in the ovary was also observed in mifepristone-treated mice (FIG. 1H). Histopathology examination with hematoxylin & eosin (H&E) staining. Scale bar, 100 μm. FIG. 11 shows survival curves of DKO mice treated with mifepristone (RU486) or placebo. Mifepristone treatment at 3 mg/m for 3 months (DKO_RU_3 mg/m_3m, 21 mice, green line) significantly extends survival of DKO mice, compared with placebo-treated group (DKO_Placebo, 21 mice, blue line): median survival, 11.2 (5.9-21.3) vs. 7.3 (5.0-12.6) months of age; HR, 0.40; 95% Cl, 0.21-0.78; log-rank test, P=0.0007. Survival was further improved with a high-dose longer treatment of mifepristone (16.7 mg/m for 6 months) (DKO_RU_16.7 mg/m_6m, 21 mice, red line): median survival, 13.1 (6.6-24.3) months of age; n=21; hazard ratio (HR), 0.13; 95% Cl, 0.06-0.30; log-rank test, P<0.0001; compared with placebo-treated DKO mice. DKO_plac, DKO mice treated with a placebo; DKO_RU, DKO mice treated with mifepristone (RU486); m, month(s).

Additional embodiments, features, and advantages of the disclosure will be apparent from the following detailed description and through practice of the disclosure.

### DETAILED DESCRIPTION

The invention relates to a seminal, far-reaching discovery that high-grade serous ovarian cancers and breast cancers are prevented by administering an antiprogestin.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed.

Chemical nomenclature for compounds described herein has been derived in accordance with International Union of Pure and Applied Chemistry (IUPAC) conventions.

### Definitions

“Antibody” refers to an immunoglobulin molecule capable of specific binding to a progesterone receptor due to the presence on such molecule of a particular domain or moiety or conformation (an “epitope”). An epitope-containing molecule may have immunogenic activity, such that it elicits an antibody production response in an animal; such molecules are termed “antigens”. Epitope-containing molecules need not necessarily be immunogenic.

“Antibody” encompasses monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, immunologically active antibody fragments (e.g., antibody fragments capable of binding to an epitope, e.g., Fab fragments, Fab′ fragments, F(ab′)2 fragments, Fv fragments, fragments containing a VL and/or VH Domain, or that contain 1, 2, or 3 of the complementary determining regions (CDRs) of such VL Domain (i.e., CDR_{L}1, CDR_{L}2, and/or CDR_{L}3) or VH Domain (i.e., CDR_{H}1, CDR_{H}2, and/or CDR_{H}3)) that specifically bind an antigen, etc., bi-functional or multi-functional antibodies, disulfide-linked bispecific Fvs (sdFv), intrabodies, and diabodies, and epitope binding fragments of any of the above. In particular, the term “antibody” is intended to encompass immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Over 200 antibody-based drugs have been approved for use or are under development.

“Chimeric antibody” refers to an antibody in which a portion of a heavy and/or light chain is identical to or homologous with an antibody from one species (e.g., mouse) or antibody class or subclass, while the remaining portion is identical to or homologous with an antibody of another species (e.g., human) or antibody class or subclass, so long as they exhibit the desired biological activity. Chimeric antibodies of interest herein include “primatized” antibodies comprising variable domain antigen binding sequences derived from a non-human primate (e.g., Old World Monkey, Ape, etc.) and human constant region sequences.

“Monoclonal antibody” as used herein refers to an antibody of a population of substantially homogeneous antibodies, i.e., the individual antibodies comprising the population are identical except for possible antibodies possessing naturally occurring mutations that may be present in minor amounts, and the term “polyclonal antibody” as used herein refers to an antibody obtained from a population of heterogeneous antibodies. The term “monoclonal” indicates the character of the antibody as being a substantially homogeneous population of antibodies, and is not to be construed as requiring production of the antibody by any particular method (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells may allow antigens to be better detected than ruptured cells by the immunized animal. Use of denaturing or harsh adjuvants, e.g., Freud's adjuvant, may rupture cells and therefore is discouraged. The immunogen may be administered multiple times at periodic intervals such as, bi-weekly, or weekly, or may be administered in such a way as to maintain viability in the animal (e.g., in a tissue recombinant). Alternatively, existing monoclonal antibodies and any other equivalent antibodies that are specific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody.

“Humanized antibody” refers to a chimeric molecule, generally prepared using recombinant techniques, having an antigen-binding site of an immunoglobulin from a non-human species and a remaining immunoglobulin structure of the molecule that is based upon the structure and/or sequence of a human immunoglobulin. The antigen binding site may comprise either complete variable domains fused onto constant domains or only the CDRs grafted onto appropriate framework regions in the variable domains. Antigen-binding sites may be wild-type or modified by one or more amino acid substitutions. This eliminates the constant region as an immunogen in human individuals, but the possibility of an immune response to the foreign variable region remains (LoBuglio, A. F. et al (1989) “Mouse/Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the variable regions as well so as to reshape them as closely as possible to human form. It is known that the variable regions of both heavy and light chains contain three CDRs which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from a non-human antibody on the FRs present in the human antibody to be modified. Application of this approach to various antibodies has been reported by Sato, K. et al. (1993) “Reshaping A Human Antibody To Inhibit The Interleukin 6-Dependent Tumor Cell Growth,” Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p18her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five or six) that are altered in their amino acid sequence(s) relative to the original antibody, which are also termed one or more CDRs “derived from” one or more CDRs from the original antibody (i.e., derived from such CDRs, derived from knowledge of the amino acid sequences of such CDRs, etc.). A polynucleotide sequence that encodes the variable domain of an antibody may be used to generate such derivatives and to improve the affinity, or other characteristics of such antibodies. The general principle in humanizing an antibody involves retaining the basic sequence of the antigen-binding site of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

Natural antibodies (such as IgG antibodies) are composed of two Light Chains complexed with two Heavy Chains. Each light chain contains a Variable Domain (VL) and a Constant Domain (CL). Each heavy chain contains a Variable Domain (VH), three Constant Domains ( CH1, CH2 and CH3), and a “Hinge” Domain (“H”) located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Domain. Thus, the structure of the light chains of an IgG molecule is n-VL-CL-c and the structure of the IgG heavy chains is n-VH-CH1-H-CH2-CH3-c (where n and c represent, respectively, the N-terminus and the C-terminus of the polypeptide). The ability of an antibody to bind an epitope of an antigen depends upon the presence and amino acid sequence of the antibody's VL and VH Domains. Interaction of an antibody light chain and an antibody heavy chain and, in particular, interaction of its VL and VH Domains forms one of the two antigen-binding sites of a natural antibody. Natural antibodies are capable of binding to only one epitope species (i.e., they are monospecific), although they can bind multiple copies of that species (i.e., exhibiting bivalency or multivalency). The Variable Domains of an IgG molecule consist of the complementarity determining regions (CDR), which contain the residues in contact with epitope, and non-CDR segments, referred to as framework segments (FR), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of an antibody Light Chain are herein respectively designated CDR_{L}1 Domain, CDR_{L}2 Domain, and CDR_{L}3 Domain. Similarly, polypeptides that are (or may serve as) the first, second and third CDR of an antibody heavy chain are herein respectively designated CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or a diabody or a single-chain binding molecule (e.g., an scFv, a BiTe, etc.), or is another type of protein. “Antigen-Binding Domain” refers to that portion of an antigen-binding molecule that is responsible for the ability of such molecule to specifically bind an epitope of an antigen. An antigen-binding fragment may contain 1, 2, 3, 4, 5 or all 6 of the CDR Domains of such antibody and, although capable of specifically binding to such epitope, may exhibit a specificity, affinity or selectivity toward such epitope that differs from that of such antibody. Preferably, however, an antigen-binding fragment will contain all 6 of the CDR Domains of such antibody. An antigen-binding fragment of an antibody may be a single polypeptide chain (e.g., an scFv), or may comprise two or more polypeptide chains, each having an amino terminus and a carboxy terminus (e.g., a diabody, a Fab fragment, an F(ab′)₂ fragment, etc.).

“Antiprogestin” means a compound or antibody: (i) that prevents progesterone from mediating its biological effects in the body, such as by blocking the activation of the progesterone receptor (PR) (i.e., PR antagonists) and/or inhibiting progesterone production or (ii) that blocks or inhibits the action or synthesis of the hormone progesterone. Examples of antiprogestins include antibodies to progesterone receptors—and selective progesterone receptor modulators (SPRMs), such as mifepristone ((8S,11R,13S,14S,17S)-11-[4-(dimethylamino)phenyl]-17-hydroxy-13-methyl-17-prop-1-ynyl-1,2,6,7,8,11,12,14,15,16-decahydrocyclopenta[a]phenanthren-3-one), ulipristal acetate ([(8S,11R,13S,14S,17R)-17-acetyl-11-[4-(dimethylamino)phenyl]-13-methyl-3-oxo-1,2,6,7,8,11,12,14,15,16-decahydrocyclopenta[a]phenanthren-17-yl] acetate), asoprisnil, Org33628, onapristone, telapristone acetate, and vilaprisan. With type I SPRMs, such as onapristone, an antagonist-bound PR does not bind to DNA. With type II SPRMs, such as mifepristone, the complex does bind to DNA. Interestingly, type II SPRMs act as agonists if the cells are stimulated with activators of the cAMP/PKA pathway; however, this effect occurs in a PR-B tissue- and species-specific manner. PRs bound to type III modulators bind DNA and have a purely antagonistic effect, even in the presence of activated PKA. This class of SPRMs includes lonaprisan. Type I, Type II, and Type III SPRM antiprogestins are included. Antiprogestins include pharmaceutically acceptable salts of the antiprogestin.

“Cancer” refers to or describes the physiological condition in patients that is typically characterized by unregulated cell growth that originate in tissues of the ovary or the breast. Included in this definition are malignant cancers. The most common type of ovarian cancer arises from epithelial cells on the surface of the ovary, and can often spread to any surface within the abdominal cavity including the fallopian tubes and peritoneal cavity. Fallopian tube cancer and primary peritoneal cancer are histologically equivalent diseases to epithelial ovarian cancer. Ovarian and breast cancers are classified from Stage I to Stage IV. Advanced ovarian or breast cancer falls within Stages III and IV; Stage III denotes cancer that is locally advanced and Stage IV denotes that distant metastasis to other body organs such as the liver and lungs has occurred. Serous ovarian carcinomas are further divided into low-grade (type I) and high-grade (type II) serous ovarian carcinomas (LGSOC and HGSOC, respectively). Most deaths are attributable to HGSOC which is approximately 20 times more common than LGSOC. High-grade serous ovarian carcinoma (HGSOC), also called high-grade serous ovarian cancer (HGSOC), is also known as high-grade serous carcinoma (HGSC) or high-grade pelvic (nonuterine) serous carcinoma.

“Chemotherapy” refers to the administration of one or more anti-cancer drugs or chemotherapeutic agents to a subject in need thereof, wherein the chemotherapeutic agents may be cytotoxic agents that may reduce the cell division of malignant cells or induce apoptosis in malignant cells. Non-limiting examples for chemotherapeutic agents for treatment of breast cancer include cyclophosphamide, methotrexate, 5-fluoruracil, doxorubicin, taxanes, or an mTOR inhibitor such as, for example, rapamycin; those for treatment of ovarian cancer include platinum-based agents (e.g., cisplatin and carboplatin) or taxane drugs (paclitaxel and docetaxel).

“Targeted therapy” refers to the administration of a compound or an antibody that specifically blocks or inhibits the activity of a targeted protein, which produces therapeutic effects in cancer treatment. Examples of targeted therapy includes tamoxifen, aromatase inhibitors (e.g., letrozole, anastrozole, and exemestane), angiogenesis inhibitors (e.g., bevacizumab), or poly (ADP-ribose) polymerase (PARP) inhibitors (e.g., olaparib, rucaparib, and niraparib).

“Genetic status” means the presence, absence, or extent/level of some physical, chemical, or genetic characteristic of the gene or its expression product(s) that is cancer-predictive. Such characteristics include, but are not limited to, mutations, copy number variants (CNVs), methylation, expression levels, activity levels, etc. These may be assayed directly (e.g., by assaying a gene's expression level) or determined indirectly (e.g., assaying the level of a gene or genes whose expression level is correlated to the expression level of the gene of interest). Those skilled in the art are familiar with various techniques for determining the status of a gene or protein including, but not limited to, whole genome or gene-specific sequencing, locus-specific genotyping (e.g., SNP arrays), large-rearrangement analysis, CNV analysis, microarray mRNA expression analysis, quantitative real-time PCR (qRT-PCR, e.g., TaqMan), immunoanalysis (e.g., ELISA, immunohistochemistry), etc. The methods of the invention may be practiced independent of the particular technique used. In some embodiments, multiple techniques are used to confirm a gene's status.

“Hormone therapy” or “endocrine therapy” refers to the administration of a compound that modulates the biological activity of human estrogen, human progesterone or their receptors to a subject in need thereof, such as receptor antagonists or receptor inhibitors. “Hormone therapy” or “endocrine therapy” includes contraceptive use of hormones, including the combined estrogen and progestin pill, the progestin-only pill, and the progestin-releasing intrauterine device. “Hormone therapy” or “endocrine therapy” also refers to the administration of hormones, which includes estrogen alone or estrogen and progestin together.

“Human” refers to a subject of both genders and at any stage of development (i.e. juvenile, adolescent, adult).

“Malignancy” or “malignant tumor” refers to the ability of a tumor to penetrate the basal membrane, invade neighboring tissues or spread through the body. A malignant tumor is synonymous with a malignant neoplasm or cancer, in particular with invasive cancer.

“Tumor” refers to a neoplasm or a lesion that is formed by an abnormal growth of neoplastic cells. The tumor can be benign, premalignant or malignant. The classification of a tissue biopsy samples from a human mammary carcinoma is preferred. The term benign lesion or tumor refers to a tumor that lacks the ability to metastasize.

“Primary tumor” refers to a tumor originating from the same tissue type as surrounding organ or tissue.

“Maintenance therapy” refers to a treatment to prevent or to delay a relapse of high-grade serous ovarian cancer or breast cancer when the cancer is in complete remission or when clinical response is achieved by a preceding therapy.

“Molecular subtype” refers to defined expression profile or pattern of a breast cancer or mammary tumor, or tumor cells thereof, where different subtypes are distinguishable upon the expression profile or pattern of a breast cancer or mammary tumor, or tumor cells thereof.

“Luminal A subtype” refers to a tumor comprising cancer cells, wherein the cancer cells are positive for the expression of the estrogen receptor and the progesterone receptor and are negative for the expression of HER2 with low expression levels of the protein Ki-67.

“Luminal B subtype” refers to a tumor comprising cancer cells, wherein the cancer cells are positive for the expression of the estrogen receptor and the progesterone receptor, and either HER2 positive or HER2 negative with high levels of Ki-67.

“HER2-enriched subtype” refers to a tumor comprising cancer cells, wherein the cancer cells are negative for the expression of the estrogen receptor and the progesterone receptor, and positive for HER2.

“Basal-like subtype” refers to a tumor comprising cancer cells that express basal markers, such as cytokeratin 5/6, cytokeratin 14 (KRT14), cytokeratin 17 (KRT17), or EGFR (HER1) or a combination of these markers, and often lack the expression of the estrogen receptor, the progesterone receptor, and HER2.

Triple-negative subtype refers to a tumor comprising cancer cells negative for the expression of the estrogen receptor, the progesterone receptor, and HER2.

“Overall survival time” means the estimated time period for which a subject will survive after the start of antiprogestin therapy. In embodiments of the invention, overall survival time is at least 5 years, e.g., 5-year survival, 6-year survival, 7-year survival, 8-year survival, 9-year survival, or at least 10-year survival.

“Pharmaceutically acceptable salts” includes derivatives of the disclosed antiprogestins, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, and nitric; other acceptable inorganic salts include metal salts such as sodium salt, potassium salt, and cesium salt; and alkaline earth metal salts, such as calcium salt and magnesium salt; and combinations comprising one or more of the foregoing salts. Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC(CH₂)ₙCOOH where n is 0-4; organic amine salts such as triethylamine salt, pyridine salt, picoline salt, ethanolamine salt, triethanolamine salt, dicyclohexylamine salt, N,N′-dibenzylethylenediamine salt; and amino acid salts such as arginate, asparginate, and glutamate, and combinations comprising one or more of the foregoing salts.

“Prevent,” “preventing,” and/or “prevention” refer to preventing the onset of a first occurrence of high-grade serous ovarian cancer or breast cancer, or preventing a second occurrence, i.e., a first recurrence, of a high-grade serous ovarian cancer or breast cancer. For prophylactic benefit, for instance, the antiprogestin may be administered to a patient at risk of developing an ovarian cancer or breast cancer to minimize the likelihood that the patient will develop the ovarian or breast cancer, or the antiprogestin may be administered to a patient to delay the onset of the ovarian or breast cancer, or the antiprogestin may be administered to completely prevent the development of the ovarian or breast cancer. The terms “prevent,” “preventing,” “prevention,” “suppress,” “suppressing,” and “suppression” refer to administering an antiprogestin either alone or as contained in a pharmaceutical composition before the onset of clinical symptoms of the cancer so as to prevent any symptom, aspect or characteristic of the cancer. For example, an antiprogestin is administered while high-grade serous ovarian cancer is undetectable by transvaginal ultrasound or by CA-125 blood test while breast cancer is undetectable by mammography or biopsy. Such preventing and suppressing need not be absolute to be useful.

“Prevent,” “preventing,” and/or “prevention” do not refer to restraining, slowing, stopping, or reversing the progression or severity of an already existing diagnosed, detected, high-grade serous ovarian cancer or breast cancer in the subject.

“Progesterone receptor” means the cellular protein also known as NR3C3 or nuclear receptor subfamily 3, group C, member 3. In humans, progesterone receptor is encoded by a single PGR gene residing on chromosome 11q22, it has two isoforms, PR-A and PR-B, that differ in their molecular weight. The PR-B is the positive regulator of the effects of progesterone, while PR-A serves to antagonize the effects of PR-B. The PR sequence is publicly available from, for example, GenBank (for example, accession numbers NP_000917.3, AAA60081.1.AAS00096.1 (protein), NM_001202474.1, NM_000926.4 (nucleotide)).

“Risk status” refers to biological or environmental influences that are known risk factors associated with a cancer. These cancer risk factors include, but are not limited to, a family history of cancer (e.g., breast cancer), age, weight, sex, history of smoking tobacco, environmental factors (e.g., exposure to asbestos, exposure to radiation, etc.), occupational risk factors (e.g., coal miner, hazmat worker, etc.), genetic factors and mutations, and so forth. It is understood that these cancer risk factors, have value either individually or in combination.

“Radiation therapy” refers to the application of ionizing radiation to a tissue that comprises or is suspected to comprise a tumor to control or kill malignant cells.

“Small molecule” refers to a low molecular weight organic compounds that may help regulate a biological process. Small molecules include any molecules with a molecular weight of about 2000 daltons or less, preferably of about 500 to about 900 daltons or less. These compounds can be natural or artificial. Biopolymers such as nucleic acids and proteins, and polysaccharides (such as starch or cellulose) are not small molecules--though their constituent monomers, ribo- or deoxyribonucleotides, amino acids, and monosaccharides, respectively, are often considered small molecules. Small molecules include pharmaceutically acceptable salts of small molecules.

“Subject” means a vertebrate, including a mammal, such as a human patient. Mammals include, but are not limited to, humans, farm animals, sport animals, and pets.

“Therapeutically effective amount” refers to the amount or dose of a progesterone antagonist, or a pharmaceutically acceptable salt thereof, upon administration to the patient, provides the desired effect in the patient under diagnosis or treatment. In determining the effective amount for a patient, a number of factors are considered by the attending diagnostician, including, but not limited to the patient's size, age, and general health; the degree of or involvement or the severity of the ovarian or breast cancer; the response of the individual patient; the particular progesterone antagonist administered; the mode of administration; the bioavailability characteristics of the preparation administered; the dose regimen selected; the use of concomitant medication; and other relevant circumstances.

The optimal dose of the antiprogestin for prevention of cancer can be determined empirically for each subject using known methods and will depend upon a variety of factors, including the activity of the antiprogestin; the age, body weight, general health, gender and diet of the subject; the time and route of administration; and other medications the subject is taking. Optimal dosages may be established using routine testing and procedures that are well known in the art.

The amount of antiprogestin that may be combined with the carrier materials to produce a single dosage form will vary depending upon the individual treated and the particular mode of administration. A physician or veterinarian having ordinary skill in the art can readily determine and prescribe the effective amount of the pharmaceutical composition required. For example, the physician or veterinarian could start doses of the antiprogestin employed in the pharmaceutical composition at levels lower than that required to achieve the desired therapeutic effect and gradually increase the dosage until the desired effect is achieved.

In general, a suitable daily dose of an antiprogestin will be that amount of the compound that is the lowest dose effective to produce a therapeutic effect. Such an effective dose will generally depend upon the factors described above and is readily determined by one having skill in the art.

Generally, therapeutically effective doses of the antiprogestin for a patient, when used for the indicated effects, will range from about 0.0001 to about 1000 mg per kilogram of body weight per day, such as from about 0.01 to about 50 mg per kg per day. If desired, the effective daily dose of the active compound may be administered as two, three, four, five, six or more sub-doses administered separately at appropriate intervals throughout the day, optionally, in unit dosage forms. In some embodiments, the dose of the antiprogestin is a low dose. Low dose refers to embodiments when the weight of the antiprogestin in unit dosage form is less than or equal to 5 mg, such as 4 mg, 3 mg, 2 mg, and 1 mg. In other embodiments, the dose of the antiprogestin is a high dose. High dose refers to embodiments when the weight of the antiprogestin in unit dosage form is greater than 10 mg, such as 25 mg, 50 mg, 75 mg, 100 mg, 200 mg, and 300 mg.

The antiprogestin and its pharmaceutically acceptable salts are generally effective over a broad dosage range. An effective dosage can be achieved by daily, weekly, or monthly administrations of a targeted dosage amount or more frequent administrations of smaller dosage amount(s). For example, in some embodiments, a 150 mg monthly dosage can be achieved by a single administration of 150 mg of the therapeutic agent per month or two administrations of 75 mg of the therapeutic agent biweekly, or the like. Dosages per month of individual agents normally fall within the range of about 0.5 mg/month to about 1000 mg/month, such as about 1 mg/month to about 500 mg/month, about 1 mg/month to about 250 mg/month, about 1 mg/month to about 150 mg/month, 1 mg/month to about 75 mg/month, and 1 mg/month to about 25 mg/month. Dosages per day of individual agents normally fall within the range of about 1 mg/month to about 5 mg/month. The antiprogestin may be used at a dose per day selected from 1 mg, 5 mg, 25 mg, and 75 mg per month. In some embodiments, the treatment period comprises a period of from about 6 months to about 10 years, more preferably from about 6 months to about 20 years. In some embodiments, the treatment period is short-term such as less than or equal to 3 years, such as 2.5 years, 2 years and 1.5 years. In other embodiments, the treatment period is long-term such as greater than 3 years, 3.5 years, 4 years, 4.5 years, 5 years, 10 years, 15 years, and 20 years.

The antiprogestin is preferably formulated as a pharmaceutical composition administered by any route that makes the compound bioavailable. The route of administration may be varied in any way, limited by the physical properties of the drugs and the convenience of the patient and the caregiver. Preferably, the antiprogestin is formulated for oral or parenteral administration including intravenous or subcutaneous administration. Such pharmaceutical compositions and processes for preparing same are well known in the art. (See, e.g., Remington: The Science and Practice of Pharmacy (D. B. Troy, Editor, 21st Edition, Lippincott, Williams & Wilkins, 2006).

Pharmaceutical compositions for use in the present invention can be in the form of sterile, non-pyrogenic liquid solutions or suspensions, coated capsules, suppositories, lyophilized powders, transdermal patches or other forms known in the art. Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono or di glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

To prolong the effect of an antiprogestin, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered antiprogestin form may be accomplished by dissolving or suspending the drug in an oil vehicle. Injectable depot forms are made by forming microencapsule matrices of the drug in biodegradable polymers such as polylactide polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the antiprogestin in liposomes or microemulsions, which are compatible with body tissues.

Compositions for rectal or vaginal administration are preferably suppositories which can be prepared by mixing the antiprogestin with suitable non-irritating excipients or carriers such as cocoa butter, polyethylene glycol or a suppository wax which are solid at ambient temperature but liquid at body temperature and therefore melt in the rectum or vaginal cavity and release the antiprogestin.

Solid dosage forms for oral administration include capsules, tablets, pills, powders, and granules. In such solid dosage forms, the antiprogestin is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the antiprogestin(s) only, or preferentially, in a certain part of the intestinal tract, optionally, in a delayed manner. Examples of embedding compositions that can be used include polymeric substances and waxes.

The antiprogestin can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the antiprogestin may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the antiprogestin, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of antiprogestins include ointments, pastes, creams, lotions, gels, powders, solutions, sprays, inhalants or patches. The antiprogestin is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an antiprogestin, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Aerosolized formulations of the invention may be delivered using an aerosol forming device, such as a jet, vibrating porous plate or ultrasonic nebulizer, preferably selected to allow the formation of an aerosol particles. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, Calif.), SIDESTREAM nebulizers (Medic Aid Ltd., West Sussex, England), PARI LC and PARI LC STAR jet nebulizers (Pari Respiratory Equipment, Inc., Richmond, Va.), and AEROSONIC (DeVilbiss Medizinische Produkte (Deutschland) GmbH, Heiden, Germany) and ULTRAAIRE (Omron Healthcare, Inc., Vernon Hills, Ill.) ultrasonic nebulizers.

Antiprogestins of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. The compounds of the present invention can also be administered in the form of liposomes. As is known in the art, liposomes are generally derived from phospholipids or other lipid substances. Liposomes are formed by mono or multi lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

When the subject in need is at risk of suffering from high-grade serous ovarian cancer or breast cancer, the subject can be treated with an antiprogestin in any combination with one or more other agents. In some embodiments, the subject is treated with an antiprogestin in combination with one or more of the other agents used in maintenance therapy. Agents used in maintenance therapy include, for example, anti-cancer agents and anti-neoplastic agents. Specific examples include paclitaxel and PARP inhibitors. In embodiments of the invention, antiprogestins are also administered in combination with a hormone therapy.

The invention of the present disclosure can be described as embodiments in any of the following enumerated clauses. It will be understood that any of the embodiments described herein can be used in connection with any other embodiments described herein to the extent that the embodiments do not contradict one another.

Clause 1. A method to prevent an occurrence of a high-grade serous ovarian cancer in a subject, the method comprising the step of administering a therapeutically effective amount of an antiprogestin to the subject.

Clause 2. A method according to the preceding clause, where the occurrence being prevented is the first occurrence.

Clause 3. A method according to any of the preceding clauses, where the occurrence being prevented is the second occurrence.

Clause 4. A method according to any of the preceding clauses, where the high-grade serous ovarian cancer is selected from the group consisting of non-metastasized high-grade ovarian cancer and metastasized high-grade ovarian cancer.

Clause 5. A method according to any of the preceding clauses, where the subject has not been diagnosed with high-grade serous ovarian cancer.

Clause 6. A method according to any of the preceding clauses, where the ovarian cancer is undetectable by biopsy in the subject when the antiprogestin administration begins.

Clause 7. A method according to any of the preceding clauses, where the high-grade serous ovarian cancer is undetectable by transvaginal ultrasound or a CA-125 blood test in the subject when the antiprogestin administration begins.

Clause 8. A method according to any of the preceding clauses, where the antiprogestin is administered to the subject to minimize the probability that the subject develops high-grade serous ovarian cancer.

Clause 9. A method according to any of the preceding clauses, where administering the antiprogestin extends overall survival time of the subject.

Clause 10. A method according to any of the preceding clauses, where an absence of high-grade serous ovarian cancer is monitored by measuring one or more biomarkers.

Clause 11. A method according to any of the preceding clauses, where the subject is a human female.

Clause 12. A method according to any of the preceding clauses, where the subject is a human female and the female is a pre-menopausal female or a post-menopausal female.

Clause 13. A method according to any of the preceding clauses, where the subject is a human female and the human female subject has a first-degree relative diagnosed with ovarian cancer, a mother diagnosed with ovarian cancer, a sister diagnosed with ovarian cancer, or a daughter diagnosed with ovarian cancer.

Clause 14. A method according to any of the preceding clauses, where the subject has a genetic status that is ovarian cancer-predictive.

Clause 15. A method according to any of the preceding clauses, where the subject has a risk status that is ovarian cancer-predictive.

Clause 16. A method according to any of the preceding clauses, where the subject is positive for a BRCA1 or 2 mutation.

Clause 17. A method according to any of the preceding clauses, where the subject does not have a BRCA1 or 2 mutation.

Clause 18. A method according to any of the preceding clauses, where the subject does not have breast cancer.

Clause 19. A method according to any of the preceding clauses, where the antiprogestin is an antibody to a progesterone receptor.

Clause 20. A method according to any of the preceding clauses, where the antiprogestin is a small molecule antiprogestin.

Clause 21. A method according to any of the preceding clauses, where the antiprogestin is a selective progesterone receptor modulator.

Clause 22. A method according to any of the preceding clauses, where the selective progesterone receptor modulator is a Type II selective progesterone receptor modulator.

Clause 23. A method according to any of the preceding clauses, where the antiprogestin is (8S,11R,13S,14S,17S)-11-[4-(dimethylamino)phenyl]-17-hydroxy-13-methyl-17-prop-1-ynyl-1,2,6,7,8,11,12,14,15,16-decahydrocyclopenta[a]phenanthren-3-one.

Clause 24. A method according to any of the preceding clauses, where the antiprogestin is [(8S,11R,13S,14S,17R)-17-acetyl-11-[4-(dimethylamino)phenyl]-13-methyl-3-oxo-1,2,6,7,8,11,12,14,15,16-decahydrocyclopenta[a]phenanthren-17-yl] acetate.

Clause 25. A method according to any of the preceding clauses, where the antiprogestin is administered to the subject in a frequency ranging from once daily to once monthly.

Clause 26. A method according to any of the preceding clauses, where the antiprogestin is administered in an amount ranging from about 0.5 mg to about 1000 mg.

Clause 27. A method according to any of the preceding clauses, where the antiprogestin is administered as a low-dose.

Clause 28. A method according to any of the preceding clauses, where the antiprogestin is administered as a high-dose.

Clause 29. A method according to any of the preceding clauses, where the antiprogestin is administered to the subject short-term.

Clause 30. A method according to any of the preceding clauses, where the antiprogestin is administered to the subject long-term.

Clause 31. A method according to any of the preceding clauses, where the antiprogestin is administered to the subject for a duration ranging from about 6 months to about 10 years.

Clause 32. A method according to any of the preceding clauses, where the antiprogestin is administered to the subject for a duration ranging from about 6 months to about 20 years.

Clause 33. A method according to any of the preceding clauses, where the administered antiprogestin obviates a need for a subsequent targeted therapy, chemotherapy and/or hormone therapy.

Clause 34. A method to prevent an occurrence of a breast cancer in a subject, the method comprising the step of administering a therapeutically effective amount of an antiprogestin to the subject.

Clause 35. A method according to clause 34, where the occurrence being prevented is the first occurrence.

Clause 36. A method according to clause 34, where the occurrence being prevented is the second occurrence.

Clause 37. A method according to any of clauses 34-36, where the breast cancer is selected from the group consisting of non-metastasized breast cancer and metastasized breast cancer.

Clause 38. A method according to any of clauses 34-37, where the breast cancer is triple-negative breast cancer.

Clause 39. A method according to any of clauses 34-38, where the subject has not been diagnosed with breast cancer.

Clause 40. A method according to any of clauses 34-39, where the breast cancer is undetectable by mammography or biopsy in the subject when the antiprogestin administration begins.

Clause 41. A method according to any of clauses 34-40, where administering the antiprogestin extends overall survival time of the subject.

Clause 42. A method according to any of clauses 34-41, where the antiprogestin is administered to the subject to minimize the probability that the subject develops breast cancer.

Clause 43. A method according to any of clauses 34-42, where an absence of breast cancer is monitored by measuring one or more biomarkers.

Clause 44. A method according to any of clauses 34-43, where the subject is a human female.

Clause 45. A method according to any of clauses 34-44, where the subject is a human female and the female is a pre-menopausal female or a post-menopausal female.

Clause 46. A method according to any of clauses 34-45, where the subject is a human female and the human female subject has a first-degree relative diagnosed with breast cancer, a mother diagnosed with breast cancer, a sister diagnosed with breast cancer, or a daughter diagnosed with breast cancer.

Clause 47. A method according to any of clauses 34-46, where the subject has a genetic status that is breast cancer-predictive.

Clause 48. A method according to any of clauses 34-47, where the subject has a risk status that is breast cancer-predictive.

Clause 49. A method according to any of clauses 34-48, where the subject is positive for a BRCA1 or a BRCA2 mutation.

Clause 50. A method according to any of clauses 34-48, where the subject does not have a BRCA1 or 2 mutation.

Clause 51. A method according to any of clauses 34-50, where the subject does not have ovarian cancer.

Clause 52. A method according to any of clauses 34-51, where the antiprogestin is an antibody to a progesterone receptor.

Clause 53. A method according to any of clauses 34-51, where the antiprogestin is a small molecule antiprogestin.

Clause 54. A method according to any of clauses 34-51, where the antiprogestin is a selective progesterone receptor modulator.

Clause 55. A method according to any of clauses 34-51 and 54, where the selective progesterone receptor modulator is a Type II selective progesterone receptor modulator.

Clause 56. A method according to any of clauses 34-51, where the antiprogestin is (8S,11R,13S,14S,17S)-11-[4-(dimethylam ino)phenyl]-17-hydroxy-13-methyl-17-prop-1-ynyl-1,2,6,7,8,11,12,14,15,16-decahydrocyclopenta[a]phenanthren-3-one.

Clause 57. A method according to any of clauses 34-51, where the antiprogestin is [(8S, 11R,13S, 14S, 17R)-17-acetyl-11-[4-(dimethylamino)phenyl]-13-methyl-3-oxo-1,2,6,7,8,11,12,14,15,16-decahydrocyclopenta[a]phenanthren-17-yl] acetate.

Clause 58. A method according to any of clauses 34-57, where the antiprogestin is administered to the subject in a frequency ranging from once daily to once monthly.

Clause 59. A method according to any of clauses 34-58, where the antiprogestin is administered in an amount ranging from about 0.5 mg to about 1000 mg.

Clause 60. A method according to any of clauses 34-59, where the antiprogestin is administered as a low-dose.

Clause 61. A method according to any of clauses 34-59, where the antiprogestin is administered as a high-dose.

Clause 62. A method according to any of clauses 34-61, where the antiprogestin is administered to the subject short-term.

Clause 63. A method according to any of clauses 34-61, where the antiprogestin is administered to the subject long-term.

Clause 64. A method according to any of clauses 34-63, where the antiprogestin is administered to the subject for a duration ranging from about 6 months to about 10 years.

Clause 65. A method according to any of clauses 34-64, where the antiprogestin is administered to the subject for a duration ranging from about 6 months to about 20 years.

Clause 66. A method according to any of clauses 34-65, where the administered antiprogestin obviates a need for a subsequent radiation therapy, targeted therapy, chemotherapy and/or hormone therapy.

### METHODS

Generation of DKO and cPR-DKO mice. DKO mice (Dicer1^{flox/flox} Pten flox/flox Amhr2^{cre/+}) were generated. A mouse line carrying Pgr^{flox/flox}, a conditional progesterone receptor (PR) gene (cPR), was provided by Drs. John Lydon and Francesco DeMayo. Conditional PR (Pgr^{flox/flox})) mice were crossed with DKO mice, generating cPR- DKO mice (Pgr^{flox/flox}Dicer1^{flox/flox} Pten^{flox/flox} Amhr2^{cre/+}). These Dicer1-Pten double-knockout (DKO) mice developed metastatic HGSCs arising from the fallopian tube with 100% penetrance. Like human ovarian cancer, this mouse HGSC spreads along the peritoneal lining across the peritoneal cavity—most notably to the omentum, as well as to the diaphragm, mesentery, and peritoneal surfaces—accompanied by ascites. All DKO mice die of widespread peritoneal metastases. Besides mirroring the clinical features of human HGSC, these mouse HGSCs closely resemble human HGSC with histopathological, molecular, and genomic similarities. Mouse use and experiments were approved by the Institutional Animal Care and Use Committee (IACUC) at Indiana University School of Medicine.

Human samples. De-identified human specimens from risk-reducing salpingo-oophorectomy (RRSO) and HGSC samples of BRCA1 mutation carriers were provided from the University of Kansas Cancer Center (KUCC)'s Biospecimen Repository Core Facility (BRCF) at the University of Kansas Medical Center (KUMC) along with relevant clinical information. Sections of formalin-fixed paraffin-embedded (FFPE) tissue specimens were obtained from women enrolled under the repository's Institutional Review Board (IRB) approved protocol (HSC #5929) and following U.S. Common Rule. Human specimens including HGSC samples were collected in accordance with IRB approval at Keimyung University School of Medicine (South Korea).

Mouse bilateral ovariectomy. To examine the effect of ovary on HGSC development in the fallopian tube, both ovaries were surgically excised with the fallopian tubes remaining intact in DKO mice. The surgery was performed at 5-6 weeks of age, when there is no sign of tumors in the fallopian tube. These ovary-deficient DKO mice were then monitored for tumor development. Another set of DKO mice without surgery served as controls. Prior to surgery, DKO mice were anesthetized using Avertin (2,2,2,-tribromoethano) (125-250 mg/kg) (Sigma-Aldrich, St. Louis, Missouri, USA). For analgesia, Carprofen (5-10 mg/kg) (TCI America, Portland, OR, USA) was used a one dose pre-operatively and every 12 hours thereafter for 24-48 hours post-operatively.

Hormone pellet implantation. To evaluate the effects of steroid hormones on HGSC development, upon ovariectomy at 5-6 weeks of age, DKO mice were implanted subcutaneously with a pellet of 17-β estradiol (E2) (0.72 mg/90 days/mouse), progesterone (P4) (25 mg/90 days/mouse), or combined E2 (0.72 mg) & P4 (25 mg). Also, for shorter periods of P4 exposure, ovariectomized DKO mice were treated with a P4 pellet of 2 mg (1 week) or 6 mg (3 weeks). Another set of ovariectomized DKO mice implanted with a placebo (a pellet without hormone) served as controls. To examine whether mifepristone (RU486) inhibits HGSC development in DKO mice by blocking PR, DKO mice with intact ovaries were implanted with a mifepristone pellet once at 9 mg/90 days (3 months) or three times at 33.3 mg/60 days (i.e., a total of 6 months). For a control group, DKO mice received a placebo pellet for mifepristone. After anesthesia, a pellet was inserted subcutaneously (SQ) after a single skin incision (0.5-1.0 cm) on the back near ovary location. The skin incision was closed with either monofilament suture or wound clips. The pellets were purchased from Innovative Research of America (Sarasota, FL, USA).

Measurement of hormone levels. Blood samples were collected in serum separation tubes (BD Microtainer® Tube; 365967; BD Biosciences, San Jose, CA, USA) from DKO mice or ovariectomized DKO mice with or without hormone treatment. Serum was isolated by centrifugating blood at 14,000 rpm for 5 minutes at room temperature. Measurements of serum progesterone (P4), 17-β estradiol (E2), testosterone (T), follicle-stimulating hormone (FSH), and luteinizing hormone (LH) levels were performed using ELISA (enzyme-linked immunoassay: P4, E2, and T) or RIA (radioimmunoassay: FSH and LH) at the Center for Research Ligand Assay and Analysis Core in Reproduction at the University of Virginia.

Immunohistochemistry. Primary and metastatic tumor tissues from different experimental groups of mice as well as human fallopian tube specimens from risk-reducing salpingo-oophorectomy (RRSO) and high-grade serous carcinoma (HGSC) samples of BRCA 1-mutation carriers were used for immunohistochemical analysis. Tissues were fixed in 10% (vol/vol) formalin at RT for 24 hours, embedded in paraffin, sectioned at 5 μm, and mounted on slides (Superfrost Plus, Fisher Scientific). Hematoxylin and eosin (H&E) staining was performed on paraffin sections according to standard protocols. Immunohistochemistry was performed as described previously (Kim et al, 2012).

RNA preparation, sequencing, and analysis. Total RNA was isolated from different experimental groups of mice: normal mouse oviduct from DKO control mice (DKO-Ctrl_FT), early-stage HGSC tumors from DKO mice (DKO_ET), premalignant fallopian tubes from ovariectomized DKO mice (DKO_Ovex_FT), early-stage HGSC tumors from ovariectomized DKO mice treated with P4 (DKO_Ovex_P4 ET). RNAs from 3 different mice per each experimental group were included. Total RNA was isolated using TRIzol (Invitrogen) and purified using RNeasy MinElute Cleanup Kit (Qiagen, Germantown, MD, USA), according to manufacturer's instructions. Preliminary RNA quality and quantity were evaluated using a Nanodrop Spectrophotometer (ND-1000). RNA quality was further examined using a bioanalyzer (Agilent 2200 and 4200). Total RNA (1-1.2 μg) was used for mRNA library preparation. Library preparation was performed using the TruSeq Stranded mRNA HT Library preparation kit (IIlumina). The libraries were pooled at a concentration of 1 nM and the sequencing analysis was performed using the NextSeq 75 (IIlumina). Reads were aligned and gene counts were generated using STAR_2.5.3a. For each library, read pairs uniquely aligned to the exon regions of each gene annotated on the genome were counted using featureCounts tool of subread package v.1.6.1. Normalized counts and differential expression analysis were conducted using DESeq2 v. 1.12.3 by providing these read counts as input. Genes with FDR<=0.05 were considered significantly differentially expressed.

Ingenuity Pathway Analysis (IPA). Commercial QIAGEN's Ingenuity Pathway Analysis (IPA, QIAGEN, Redwood City, CA, USA, www.qiagen.com/ingenuity) software was employed to identify individual differentially expressed genes (DEGs) and group them into known functions, pathways, and networks predicted to be involved in P4-induced HGSC development. RNA sequencing data from DKO-Ctrl_FT, DKO_ET, DKO_Ovex_FT, and DKO_Ovex_P4_ET were used. The analysis was performed with upregulated and downregulated genes at a cutoff of 2-fold in gene expression change (P<0.05).

Statistical analysis. Statistical analyses were performed using a GraphPad Prism software (Version 8, GraphPad Software, Inc.). P values less than 0.05 were considered statistically significant. Log-rank test was used to compare survival distributions between different experimental groups of mice; Student's t-test to compare hormone levels between placebo-treated ovariectomized DKO mice and progesterone (P4)-treated ovariectomized DKO mice at 1 week, 3 weeks, and 3 months; Student's t-test to compare hormone levels between ovariectomized DKO mice (Ovex) and each experimental group of DKO mice; and Fisher's exact test to analyze PR expression in benign and malignant ovarian tumors.

Mouse model of human high-grade serous ovarian cancer. High-grade serous ovarian cancer, also known as high-grade serous carcinoma (HGSC), is the most common and deadliest type of human ovarian cancer type accounting for over 70% of all ovarian cancer deaths. It is diagnosed predominantly in advanced stages in which the cancer has already spread throughout the body. To understand the etiology and mechanism underlying HGSC development and progression, genetically engineered mice were developed, namely double-knockout (DKO) mice, by disabling two genes, Dicer1 and Pten: Dicer1^{flox/flox} Pten^{flox/flox} Amhr2 ^{cre+}. All of these DKO mice developed metastatic ovarian cancer duplicating human metastatic HGSC. Notably, these DKO mice develop metastatic ovarian cancer in a stepwise manner from a premalignant stage to an early stage before progressing to an advanced stage with metastases. Thus, this DKO mouse model offers a unique tool to understand factors and mechanisms vital to ovarian cancer development and progression.

The ovary is critical to the development of metastatic HGSC originating in the fallopian tube. The fallopian tube is the origin of high-grade serous carcinoma (HGSC) in DKO mice (Dicer1^{flox/flox}Pten^{flox/flox} Amhr2^{cre/+}). Surgical removal of both ovaries at a premalignant stage (at 4-10 weeks of age) did not prevent the fallopian tubes from developing tumors. However, absence of the ovaries significantly prolonged the survival of these mice. Ovary-deficient DKO mice lived 4.5 months (˜13.5 human years) longer than intact DKO mice (median survival, 13.2 months [6.9-17.4] vs. 8.7 months [5.9-12.4]; n=27 and 33 mice, respectively; hazard ratio [HR], 0.26; 95% confidence interval [Cl], 0.14-0.50; p<0.0001). Thus, the ovary, though not the tumor origin, may play a role in HGSC in this model.

With intact ovaries, DKO mice developed predominantly HGSC in the fallopian tube, resulting in extensive peritoneal HGSC metastases with complete penetrance. In contrast, ovariectomized DKO mice still formed fallopian tube tumors, but these primary tumors were generally large, heterogeneous, and composed mainly of cystic and fibrous tissues as well as stromal tumors with only a small segment of HGSC, accompanied by markedly reduced or little peritoneal metastasis. Thus, the ovary appears to influence the type of tumor that develops in the fallopian tube of DKO mice. Absence of the ovaries profoundly diminishes the ability of DKO mice to develop HGSC in the fallopian tube, resulting in nearly absent or sporadic peritoneal metastasis, and leads to longer survival in ovary-deficient DKO mice. Hence, presence of the ovary is vital to the development of HGSC equipped with metastatic potential.

Progesterone induces HGSC with metastatic potential in ovary-deficient DKO mice. Ovary-deficient DKO mice were treated with steroid hormones. In this experiment, upon surgical removal of the ovaries at 5-6 weeks of age (premalignant stage), ovariectomized DKO mice were implanted subcutaneously with a pellet of (i) progesterone (P4; 25 mg), (ii) 17β-estradiol (E2; 0.72 mg), (iii) P4+E2, or (iv) placebo for 3 months (m). Tumor development and mouse survival were then monitored.

With progesterone (P4) treatment alone, all ovariectomized DKO mice developed predominantly HGSC in the fallopian tube with widespread and abundant peritoneal metastases accompanied by ascites (100%: 32/32 mice). These primary and metastatic tumors were histopathologically confirmed as HGSC. P4 treatment elevated serum progesterone levels, while not affecting estrogen and testosterone levels in ovariectomized DKO mice, indicating that P4 drives the development of primary and metastatic HGSCs. In contrast, ovariectomized DKO mice treated with placebo generally formed large heterogeneous fallopian tube tumors with scant peritoneal metastasis, as observed similarly in ovariectomized DKO mice (with no placebo). Likewise, histologically, primary fallopian tube tumors from placebo-treated ovariectomized DKO mice comprised mostly non-HGSC tissues—stromal tumors and cystic, fibrous tissues—with sporadic HGSC cells.

HGSCs with extensive peritoneal metastases resulted in significantly shortened survival in P4-treated ovariectomized DKO mice compared with placebo-treated ovariectomized DKO mice (median survival, 6.5 [5.1-13.5] [n=32 mice] vs. 12.2 months of age [6.8-19.6] [n=21 mice]; HR, 3.01; 95% Cl, 1.7-5.4; p<0.0001) (FIG. 2K). These in vivo findings evidence that progesterone (P4) is the key ovarian factor enabling the development of HGSC with metastatic potential, leading to a poor prognosis and decreased survival in DKO mice.

Estrogen appeared to attenuate the effect of progesterone on HGSC development and mouse survival. Ovariectomized DKO mice treated with progesterone plus 17β-estradiol (P4+E2) were capable of developing primary and metastatic HGSCs, yet with diminished capacity. In ovariectomized DKO mice with P4+E2, primary fallopian tube HGSCs tended to be small and often heterogeneous harboring non-HGSC components, accompanied by reduced or sporadic peritoneal metastases. The attenuated development of HGSC with lessened metastatic potential tended to extend the survival of ovariectomized DKO mice receiving P4+E2 (median survival, 9.9 months of age [5.8-13.5]; n=10 mice; HR, 0.56; 95% Cl, 0.30-1.06; p=0.09) relative to P4 alone (6.5 months of age [5.1-13.5]; n=32 mice). Ovariectomized DKO mice with E2 alone did not produce any tumors in the fallopian tube, but preferentially developed tumors or swelling in the uterus (90.9%: 10/11 mice), likely owing to unopposed estrogen-induced uterine proliferation attributable to a lack of progesterone. Accordingly, estrogen may suppress HGSC development by opposing or attenuating the effects of progesterone.

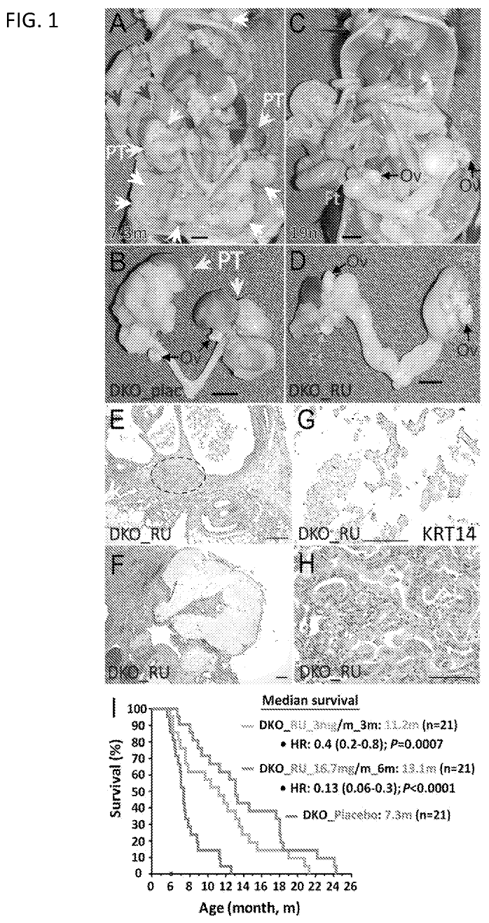

Mifepristone inhibited HGSC development and significantly extended mouse survival. Progesterone generates its biological effects through the progesterone receptors (PR). Thus, to evaluate further whether progesterone signaling is critical to the development of HGSC with metastatic potential, DKO mice were treated with the PR antagonist mifepristone (RU486), an antiprogestin, that acts by binding to PR and inhibiting the transcription of its target genes. DKO mice (with intact ovaries) at 5-6 weeks of age (premalignant stage) were implanted with mifepristone (3 mg/m) or placebo for 3 months. As expected, DKO mice treated with a placebo developed primary HGSC coupled with abundant peritoneal HGSC metastases (FIG. 1A-B). In contrast, as predicted, antiprogestin treatment effectively suppressed HGSC development in the fallopian tube and peritoneal metastasis, that was evidenced by a markedly low degree, or near absence, of peritoneal metastases (FIG. 1C-D). Histologically, the fallopian tubes from mifepristone-treated mice were composed of cystic and fibrous tissues as well as small segments of carcinoma cells in the stroma, accompanied by limited peritoneal metastasis (FIG. 1E-G).

Additionally, in some cases, low-grade endometrioid-type carcinomas were observed in the ovary, fallopian tube, or uterus (FIG. 1H). Plausibly, inhibition of progesterone signaling decreases HGSC formation in the fallopian tube and its metastasis, that leads to an increased tumorigenic propensity for another tumor type in these reproductive tissues, in which deletion of Dicerl and Pten also occurs. Also, one-third of DKO mice (33.3%: 7/21) in the low-dose (3 mg/m) group still developed fallopian tube HGSC and widespread peritoneal metastases similar to placebo-treated DKO mice. However, a high dose of mifepristone (16.7 mg/m for 6 m) effectively suppressed HGSC development and peritoneal metastasis: only 9.5% (2/21) of these DKO mice were not inhibited by mifepristone. Together, these findings evidence that the inhibition of progesterone signaling by antiprogestin decreases HGSC formation and peritoneal metastasis in DKO mice. Antiprogestin treatment markedly increases mouse survival. DKO mice treated with mifepristone (3 mg/m) lived longer by nearly 4 months (˜12 human years) than DKO mice with placebo (FIG. 11; median survival, 11.2 [5.9-21.3] vs. 7.3 months of age [5.0-12.6]; n=21 mice each; HR, 0.40; 95% Cl, 0.21-0.78; p=0.0007). Moreover, a high-dose longer treatment with mifepristone (16.7 mg/m) further extended survival (median survival, 13.1 months of age [6.6-24.3]; n=21; HR, 0.13; 95% Cl, 0.06-0.30; p<0.0001), allowing the DKO mice to live nearly 6 months (˜18 human years) longer than placebo-treated DKO mice. Thus, by blocking PR and thereby suppressing HGSC development and peritoneal metastases, antiprogestin treatment improves mouse survival.

Genetic inactivation of PR suppressed HGSC development in DKO mice. Besides a pharmacological inhibition of PR by mifepristone, the PR gene (Pgr) was genetically ablated in DKO mice to further verify the impact of PR signaling on HGSC development. To inactivate the PR gene in the fallopian tube, a conditional PR allele (Pgr^{flox/flox}) was bred into DKO mice (Dicer1^{flox/flox} Pten^{flox/flox} Amhr2^{cre/+}), generating cPR-DKO mice (Pgr^{flox/flox}Dicer1^{flox/flox} Pten^{flox/flox} Amhr2^{cre/+}).

In cPR-DKO mice, the PR gene would be deleted in the fallopian tube cells lacking Dicerl and Pten, from which HGSC arises, as well as in other reproductive tissues, including the ovary, uterus, and cervix.

The genetic ablation of the PR gene inhibited HGSC development. Approximately one-third of cPR-DKO mice (32.5%: 13/40 mice) developed ovarian or fallopian tube tumors with limited peritoneal metastasis. In cases with unambiguous fallopian tube origin of tumors, the tumors were histologically characterized as poorly differentiated tumors or carcinomas. As expected, PR inactivation did not completely block HGSC development. Similar to the low-dose antiprogestin treatment, a portion of cPR-DKO mice (32.5%: 13/40 mice) developed primary fallopian tube HGSC with widespread peritoneal metastases similar to DKO mice, suggesting that HGSC can also form independent of PR signaling. In addition to ovarian and fallopian tube primary tumors, cPR-DKO mice formed primary cervical or uterine tumors, or both, with little metastasis (55.0%: 22/40 mice). Development of cervical, uterine, and ovarian tumor phenotypes, that were not observed in mifepristone-treated DKO mice, is likely due to genetic deletion of Pgr, Dicer1, and Pten during the embryonic development driven by Amhr2-Cre. Collectively, these results show that genetic inactivation of PR suppresses HGSC development and peritoneal metastasis in DKO mice. Hence, these findings corroborate the antiprogestin results and further evidence that progesterone/PR signaling is crucial to the development of HGSC harboring metastatic potential.

Loss of PR expression in mouse and human HGSCs was observed. At a premalignant stage, it was found that PR expression was widespread in the epithelium and stroma of the fallopian tube in DKO mice. Mouse HGSCs, however, were devoid of PR expression.

Similarly, in humans, PR was abundantly expressed in the epithelium and stroma of normal as well as premalignant fallopian tubes—from risk-reducing salpingo-oophorectomy (RRSO) from BRCA1-mutation carriers (5/5 cases), who are at genetically high risk of developing HGSC. PR expression, however, was completely absent in HGSC tissues from BRCA1-mutation carriers who had developed ovarian cancer (5/5 cases). Additionally, in human HGSCs from a population with undetermined BRCA status, a majority of HGSC cases (73.7%: 84/114 cases) were negative for PR.

These results suggest that after progesterone has induced PR-expressing fallopian tube cells into HGSC, these HGSC cells then lose PR expression and that while progesterone/PR signaling is primarily involved in the initiation and formation of HGSC, it may not directly contribute to the progression of HGSC. A similar pattern of PR expression and PR loss between DKO model and BRCA1-mutation carriers postulates that progesterone/PR signaling may be critical to HGSC development in these high-risk women.

Altered BRCA1 signaling and BRCAness in P4-regulated pathways underlying HGSC development. To examine the molecular signaling pathways underlying progesterone (P4)-induced HGSC development, gene-expression profiling using RNA sequencing was performed and, subsequently, pathway analysis using genes whose expression was altered in early-stage HGSC (ET) from P4-treated ovariectomized DKO mice (P4-Ovex-DKO). Among the top 10 significant pathways for the upregulated genes, DNA damage/repair regulation and BRCA1 signaling were notably altered, suggesting that molecular mechanisms involved in P4-induced HGSC development may be linked to processes implicated in ovarian cancer (HGSC) among BRCA1-mutation carriers.

Also, non-BRCA tumors can harbor molecular features similar to those in tumors from BRCA-mutation carriers, known as “BRCAness.” Though not carrying a Brca1 or 2 mutation, P4-induced HGSC and DKO HGSC exhibit widespread molecular alterations in BRCA signaling and homologous recombination (HR) pathways, amply demonstrating “BRCAness.” Thus, HGSC-inducing role of ovarian P4 and antiprogestin prevention of HGSC could also be closely relevant to BRCA-mutation carriers, as well as women negative for BRCA mutations.

Progesterone (P4) signaling is critical to breast cancer development. Intriguingly, HGSC and triple-negative breast cancer (TNBC), albeit arising from disparate tissues, are genomically similar malignances, raising the possibility of a similar mechanism of cancer development. Therefore, our findings suggest that progesterone P4/PR signaling is critical to TNBC development and antiprogestins can prevent breast cancer.

To examine the vital importance of P4/PR signaling in breast cancer, Brca1^{flox/flox} p53 ^{LSL-R172H/+} Pten^{flox/flox} Pgr^{cre/+} mice were generated, in which Brca1, p53, and Pten were inactivated or mutated in PR-expressing cells in the mammary glands. These mice rapidly developed multiple mammary gland tumors from 5.9 to 7.2 months of age with a high penetrance rate (81.8%: 9/11 mice). Histopathological examination of these tumors confirmed that some early-stage mammary gland tumors (visually small tumors) formed ductal carcinoma in situ (DCIS), while some mammary gland tumors had both DCIS and invasive ductal carcinomas, including poorly differentiated adenocarcinoma. Crucially, these invasive tumors either lack or exhibit low levels of expression of ER, PR, and HER2 (human epidermal growth factor receptor 2), while robustly expressing basal markers such as cytokeratin 14 (KRT14) and cytokeratin 17 (KRT17), indicating that these tumors are TNBC/basal-like tumors. Together, these findings support that progesterone signaling is critical to TNBC development. Furthermore, importance of P4/PR signaling goes beyond TNBC development. The progestin (synthetic progesterone) component in hormone-replacement therapy (HRT) increases breast cancer risk not only in BRCA 1-mutation carriers (who predominantly develop TNBC)—but also in postmenopausal women in the general population. Approximately 80% of breast cancer cases are hormone-receptor-positive breast cancer in the general population, as in BRCA2 carriers. Therefore, one skilled in the art would reasonably predict that the breast cancer preventive effects of antiprogestins can be extended to general population of women and all breast cancer types.

Other variations or embodiments will be apparent to a person of ordinary skill in the art from the above-description. Thus, the foregoing embodiments are not to be construed as limiting the scope of the claimed invention. All references disclosed are expressly incorporated by reference in their entirety.

What is claimed:

1. A method to prevent an occurrence of a high-grade serous ovarian cancer in a subject, the method comprising the step of administering a therapeutically effective amount of an antiprogestin to the subject.

2. The method according to claim 1, where the occurrence being prevented is the first occurrence.

3. The method according to claim 1, where the occurrence being prevented is the second occurrence.

4. The method according to claim 1, where the high-grade serous ovarian cancer is selected from the group consisting of non-metastasized high-grade serous ovarian cancer and metastasized high-grade serous ovarian cancer.

5. The method according to claim 1, where the subject has not been diagnosed with high-grade serous ovarian cancer.

6. The method according to claim 1, where the high-grade serous ovarian cancer is undetectable by biopsy in the subject when the antiprogestin administration begins.

7. The method according to claim 1, where the high-grade serous ovarian cancer is undetectable by transvaginal ultrasound and a CA-125 blood test in the subject when the antiprogestin administration begins.

8. The method according to claim 1, where the antiprogestin is administered to the subject to minimize the probability that the subject develops high grade serous ovarian cancer.

9. The method according claim 1, where administering the antiprogestin extends overall survival time of the subject.

10. The method according to claim 1, where an absence of high-grade serous ovarian cancer is monitored by measuring one or more biomarkers.

11. The method according to claim 1, where the subject is a human female.

12. The method according claim 1, where the subject is a human female and the female is a pre-menopausal female or a post-menopausal female.

13. The method according to claim 1, where the subject is a human female and the human female subject has a first-degree relative diagnosed with ovarian cancer, a mother diagnosed with ovarian cancer, a sister diagnosed with ovarian cancer, or a daughter diagnosed with ovarian cancer.

14. The method according to claim 1, where the subject has a genetic status that is ovarian cancer-predictive.

15. The method according to claim 1, where the subject has a risk status that is ovarian cancer-predictive.

16. The method according to claim 1, where the subject is positive for a BRCA1 or 2 mutation.

17. The method according to claim 1, where the subject does not have a BRCA1 or 2 mutation.

18. The method according to claim 1, where the subject does not have breast cancer.

19. The method according to claim 1, where the antiprogestin is an antibody to a progesterone receptor.

20. The method according to claim 1, where the antiprogestin is a small molecule antiprogestin.

21. The method according to claim 1, where the antiprogestin is a selective progesterone receptor modulator.

22. The method according to claim 1, where the selective progesterone receptor modulator is a Type II selective progesterone receptor modulator.

23. The method according to claim 1, where the antiprogestin is (8S,11R,13S,14S,17S)-11-[4-(dimethylamino)phenyl]-17-hydroxy-13-methyl-17-prop-1-ynyl-1,2,6,7,8,11,12,14,15,16-decahydrocyclopenta[a]phenanthren-3-one.

24. The method according to claim 1, where the antiprogestin is [(8S,11R,13S,14S,17R)-17-acetyl-11-[4-(dimethylamino)phenyl]-13-methyl-3-oxo-1,2,6,7,8,11,12,14,15,16-decahydrocyclopenta[a]phenanthren-17-yl] acetate.

25. The method according to claim 1, where the antiprogestin is administered to the subject in a frequency ranging from once daily to once monthly.

26. The method according to claim 1, where the antiprogestin is administered in an amount ranging from about 0.5 mg to about 1000 mg. dose.

27. The method according to claim 1, where the antiprogestin is administered as a low-dose.

28. The method according to claim 1, where the antiprogestin is administered as a high-dose.

29. The method according to claim 1, where the antiprogestin is administered to the subject short-term.

30. The method according to claim 1, where the antiprogestin is administered to the subject long-term.

31. The method according to claim 1, where the antiprogestin is administered to the subject for a duration ranging from about 6 months to about 10 years.

32. The method according to claim 1, where the antiprogestin is administered to the subject for a duration ranging from about 6 months to about 20 years.

33. The method according to claim 1, where the administered progestin obviates a need for a subsequent targeted therapy, chemotherapy and/or hormone therapy.

34. A method to prevent an occurrence of a breast cancer in a subject, the method comprising the step of administering a therapeutically effective amount of an antiprogestin to the subject.

35. The method according to claim 34, where the occurrence being prevented is the first occurrence.

36. The method according to claim 34, where the occurrence being prevented is the second occurrence.

37. The method according to claim 34, where the breast cancer is selected from the group consisting of non-metastasized breast cancer and metastasized breast cancer.

38. The method according to claim 34, where the breast cancer is triple-negative breast cancer.

39. The method according to claim 34, where the subject has not been diagnosed with breast cancer.

40. The method according to claim 34, where the breast cancer is undetectable by biopsy in the subject when the antiprogestin administration begins.

41. The method according to claim 34, where administering the antiprogestin extends overall survival time of the subject.

42. The method according to claim 34, where the antiprogestin is administered to the subject to minimize the probability that the subject develops breast cancer.

43. The method according to claim 34, where an absence of breast cancer is monitored by measuring one or more biomarkers.

44. The method according to claim 34, where the subject is a human female.

45. The method according to claim 34, where the subject is a human female and the female is a pre-menopausal female or a post-menopausal female.

46. The method according to claim 34, where the subject is a human female and the human female subject has a first-degree relative diagnosed with breast cancer, a mother diagnosed with breast cancer, a sister diagnosed with breast cancer, or a daughter diagnosed with breast cancer.

47. The method according to claim 34, where the subject has a genetic status that is breast cancer-predictive.

48. The method according to claim 34, where the subject has a risk status that is breast cancer-predictive.

49. The method according to claim 34, where the subject is positive for a BRCA1 or a BRCA2 mutation.

50. The method according to claim 34, where the subject does not have a BRCA1 or 2 mutation.

51. The method according to claim 34, where the subject does not have ovarian cancer.

52. The method according to claim 34, where the antiprogestin is an antibody to a progesterone receptor.

53. The method according to claim 34, where the antiprogestin is a small molecule antiprogestin.

54. The method according to claim 34, where the antiprogestin is a selective progesterone receptor modulator.

55. The method according to claim 34, where the selective progesterone receptor modulator is a Type II selective progesterone receptor modulator.

56. The method according to claim 34, where the antiprogestin is (8S, 11R, 13S, 14S, 17S)-11-[4-(dimethylamino)phenyl]-17-hydroxy-13-methyl-17-prop-1-ynyl-1,2,6,7, 8, 11, 12, 14, 15,16-decahydrocyclopenta[a]phenanthren-3-one.

57. The method according to claim 34, where the antiprogestin is [(8S,11R,13S,14S,17R)-17-acetyl-11-[4-(dimethylam ino)phenyl]-13-methyl-3-oxo-1,2,6,7,8, 11,12,14,15,16-decahydrocyclopenta[a]phenanthren-17-yl] acetate.

58. The method according to claim 34, where the antiprogestin is administered to the subject in a frequency ranging from once daily to once monthly.

59. The method according to claim 34, where the antiprogestin is administered in an amount ranging from about 0.5 mg to about 1000 mg.

60. The method according to claim 34, where the antiprogestin is administered as a low-dose.

61. The method according to claim 34, where the antiprogestin is administered as a high-dose.

62. The method according to claim 34, where the antiprogestin is administered to the subject short-term.

63. The method according to claim 34, where the antiprogestin is administered to the subject long-term.

64. The method according to claim 34, where the antiprogestin is administered to the subject for a duration ranging from about 6 months to about 10 years.

65. The method according to claim 34, where the antiprogestin is administered to the subject for a duration ranging from about 6 months to about 20 years.

66. The method according to claim 34, where the administered antiprogestin obviates a need for a subsequent radiation therapy, targeted therapy, chemotherapy and/or hormone therapy.
